(19) United States
(12) Patent Application Publication
(10) Pub. No.: US 2019/0198168 A1
(43) Pub. Date: Jun. 27, 2019

(54) Techniques For Performing Remote Diagnosis Of A Medical Device

(71) Applicant: Stryker Corporation, Kalamazoo, MI (US)

(72) Inventors: Frank Lee, Kalamazoo, MI (US); Krishna Bhimavarapu, Kalamazoo, MI (US)

(21) Appl. No.: 16/211,932

(22) Filed: Dec. 6, 2018

Related U.S. Application Data
(60) Provisional application No. 62/609,817, filed on Dec. 22, 2017.

(51) Int. Cl. G16H 40/67; G16H 40/63; A61G 7/018; G16H 20/30; G16H 10/60

(52) U.S. Cl. CPC G16H 40/67; G16H 40/63; A61G 7/0506; G16H 20/30; G16H 10/60; A61G 7/018

(57) ABSTRACT

A system and method for remotely diagnosing a medical device are disclosed. The system includes a medical device including an actuator for moving a portion of the medical device, and a controller coupled to a communication network. The system also includes a remote user interface, which is coupled to the communication network is configured to execute a remote diagnostic application. The remote diagnostic application is configured to provide a state of the medical device. The remote user interface is further configured to receive a selected remote control function from the user, and transmit an input signal corresponding to the selected remote control function to the controller of the medical device to execute the selected remote control function based on the input signal.

### CROSS-REFERENCE TO RELATED APPLICATION

The subject patent application claims priority to and all the benefits of U.S. Provisional Patent Application No. 62/609,817 filed on Dec. 22, 2017, the disclosure of which is hereby incorporated by reference in its entirety.

### BACKGROUND

Medical devices used to provide patient care often include a variety of components and associated functions. Such medical devices may include, for example, patient support apparatuses such as hospital beds, stretchers, cots, tables, wheelchairs, recliners, and chairs for patient care. Other medical devices used in providing patient care may include equipment such as lights, televisions, temperature management systems, respirators, IV lines, surgical tools, and heart rate monitors that may be used in medical procedures or in the provision of medical services to patients. Due to the potential for harm to a patient if a medical device malfunctions and due to the harsh use conditions that the medical devices may be subjected to, medical devices may need to be monitored frequently to ensure that the medical devices are operating properly.

For example, if a patient support apparatus is not responding or responding incorrectly to a command from a patient disposed on the patient support apparatus or a caregiver adjacent to the patient support apparatus, the patient support apparatus may need to undergo diagnostics to determine a malfunction causing the improper response. As such, proper diagnosis of the patient support apparatus allows for discovery and resolution of an error of the patient support apparatus.

Typically, diagnosis of a patient support apparatus is performed within a vicinity of the patient support apparatus by individuals capable of performing such tasks. Therefore, diagnosis of the patient support apparatus depends on an availability of the individual capable of performing such tasks to be within a proximity of the patient support apparatus. As such, there are opportunities to address at least the aforementioned problems.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 is a perspective view of a system for performing remote diagnosis of a patient support apparatus including a patient support apparatus, a remote user interface, a local user interface, an image sensor, and a controller.

FIG. 2A is a schematic diagram illustrating an embodiment of the system including the controller, the remote user interface, and the image sensor;

FIG. 2B is a schematic diagram illustrating an embodiment of the system including the controller, the remote user interface, the local user interface, and the image sensor;

FIG. 3A is a view of an embodiment of the remote user interface; and

FIG. 3B is a view of an embodiment of the local user interface.

### DETAILED DESCRIPTION

Referring to the Figures, wherein like numerals indicate like or corresponding parts throughout the several views, techniques for performing remote diagnosis of a medical device are provided.

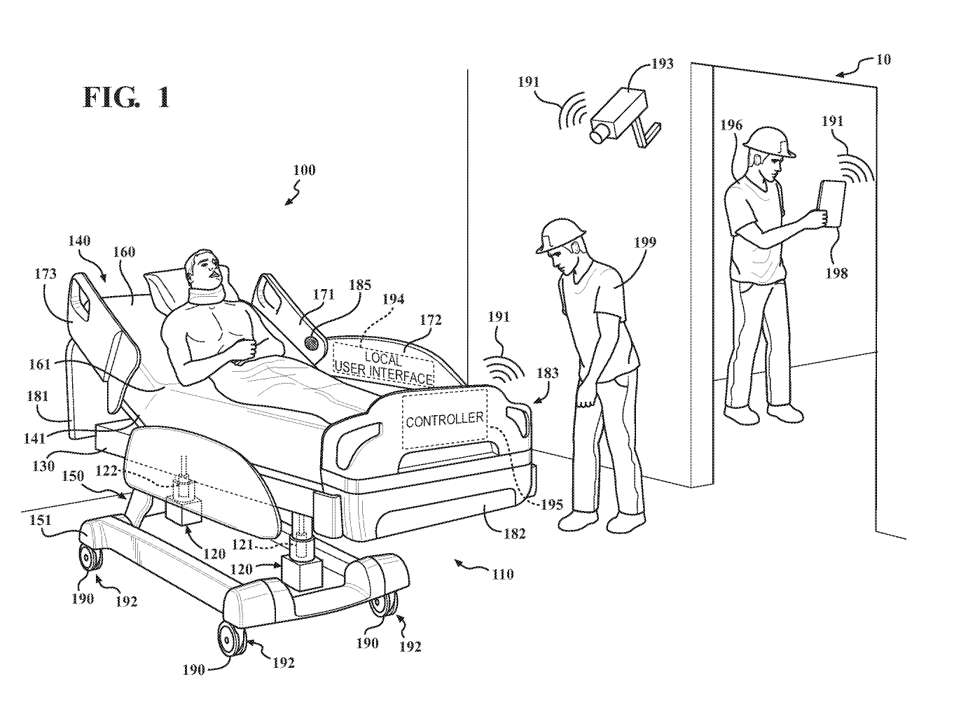

FIG. 1 illustrates a system of performing remote diagnosis of a medical device, such as a patient support apparatus. As shown, a patient support apparatus 100 for supporting a patient in a health care setting is provided. The patient support apparatus 100 illustrated in FIG. 1 includes a hospital bed. However, in other embodiments, the patient support apparatus 100 may include a stretcher, a cot, a table, a wheelchair, a recliner, a chair for patient care, or any other similar apparatus utilized in the care of a patient. While the following embodiments are described with reference to a patient support apparatus 100, it should be recognized that the embodiments may alternatively be used with any suitable medical device used to provide patient care, such as, without limitation, lights, televisions, temperature management systems, respirators, IV lines, and heart rate monitors.

A support structure 110 provides support for the patient. The support structure 110 illustrated in FIG. 1 includes a base 150 and a support frame 130. The base 150 includes a base frame 151. The support frame 130 is spaced above the base frame 151 in FIG. 1. The support structure 110 also includes a patient support deck 140 disposed on the support frame 130. The patient support deck 140 includes several sections, some of which are capable of articulating relative to the support frame 130, such as a back section, a seat section, a thigh section, and a foot section. The patient support deck 140 provides a patient support surface 141 upon which the patient is supported.

A mattress 160 may be disposed on the patient support deck 140 during use. The mattress 160 includes a secondary patient support surface 161 upon which the patient is supported. In addition, the mattress 160 may be omitted in certain embodiments, such that the patient rests directly on the patient support surface 141.

The base 150, support frame 130, patient support deck 140, and patient support surface 141 each have a head end and a foot end corresponding to a designated placement of the patient's head and feet on the patient support apparatus 100. The construction of the support structure 110 may take on any suitable design, and is not limited to that specifically set forth above.

Side rails 171, 172, 173, 174 are coupled to the support frame 130 or the patient support deck 140 and are thereby supported by the base 150. A first side rail 171 is positioned at a left head end of the patient support deck 140. A second side rail 172 is positioned at a left foot end of the support frame 130. A third side rail 173 is positioned at a right head end of the patient support deck 140. A fourth side rail 174 is positioned at a left foot end of the support frame 130. If the patient support apparatus 100 is a stretcher or a cot, there may be fewer side rails. The side rails 171, 172, 173, 174 are movable to a raised position in which they block ingress and egress into and out of the patient support apparatus 100, one or more intermediate positions, and a lowered position in which the side rails 171, 172, 173, 174 are not an obstacle to such ingress and egress. In still other configurations, the patient support apparatus 100 may not include any side rails.

A headboard 181 and a footboard 182 are coupled to the support frame 130. In other embodiments, when the headboard 181 and footboard 182 are included, the headboard 181 and footboard 182 may be coupled to other locations on the patient support apparatus 100, such as the base 150. In still other embodiments, the patient support apparatus 100 does not include the headboard 181 and/or the footboard 182.

Caregiver interfaces 183, such as handles, are shown integrated into the footboard 182 and side rails 171, 172, 173, 174 to facilitate movement of the patient support apparatus 100 over floor surfaces. Additional caregiver interfaces 183 may be integrated into the headboard 181 and/or other components of the patient support apparatus 100. The caregiver interfaces 183 are graspable by a remote user to manipulate the patient support apparatus 100 for movement.

Wheels 190 are coupled to the base 150 to facilitate transport over the floor surfaces. The wheels 190 are arranged in each of four quadrants of the base 150 adjacent to corners of the base 150. In the embodiment shown, the wheels 190 are caster wheels able to rotate and swivel relative to the support structure 110 during transport. Each of the wheels 190 forms part of a caster assembly 192. Each caster assembly 192 is mounted to the base 150. It should be understood that various configurations of the caster assemblies 192 are contemplated. In addition, in some embodiments, the wheels 190 are not caster wheels and may be non-steerable, steerable, non-powered, powered, or combinations thereof. Additional wheels are also contemplated. For example, the patient support apparatus 100 may include four non-powered, non-steerable wheels, along with one or more powered wheels. In some cases, the patient support apparatus 100 may not include any wheels.

In other embodiments, one or more auxiliary wheels (powered or non-powered), which are movable between stowed positions and deployed positions, may be coupled to the support structure 110. In some cases, when these auxiliary wheels are located between caster assemblies 192 and contact the floor surface in the deployed position, they cause two of the caster assemblies 192 to be lifted off the floor surface thereby shortening a wheel base of the patient support apparatus 100. A fifth wheel may also be arranged substantially in a center of the base 150.

As shown in FIG. 1, the system 10 may include an actuatable device 120 and actuators 121, 122. The actuators 121, 122 may be further defined as being capable of moving the actuatable device 120. The actuators 121, 122 may be coupled to the support structure 110 to move the patient when the patient is disposed on the patient support structure 110. In the embodiment of the patient support apparatus 100 shown in FIG. 1, the patient support apparatus 100 includes two actuators 121, 122. However, it is to be noted that the patient support apparatus 100 may include any suitable number of actuators 121, 122. Furthermore, any of the techniques described herein can utilize any number of actuators 121, 122 individually or in combination.

The actuators 121, 122 should be broadly understood as a type of motor or device that is capable of moving or controlling a mechanism or a system. For example, some suitable, non-limiting examples of the actuators 121, 122 are mechanical, hydraulic, pneumatic, electric, thermal, or magnetic actuators. The actuators 121, 122 may also include motors, such as a rotational or linear motor. In a further example, the actuators 121, 122 may include an inflation actuator. In sum, it should be understood that any type of actuator can be used in certain applications.

As described above, the actuators 121, 122 may be further defined as being capable of moving an actuatable device 120. These actuatable devices 120 are not particularly limited, and may include any device or system that includes one or more actuators 121, 122. In certain embodiments, the actuatable device 120 is one that, when actuated, results in a change of position of the patient support surfaces 141, 161 of the patient support structure 110. This change in position of one or more patient support surfaces 141, 161 when the patient occupies the patient support apparatus 100, results in a change in the position of one or more portions of the patient's body.

More specifically, in situations where a patient occupies the patient support apparatus 100, i.e., contacts one or more patient support surfaces 141, 161, operation of each of the actuatable devices 120 results in movement of one or more portions of the patient in one or more dimensions relative to a static surface, such as relative to a floor of a hospital. Examples of such movement include, but are not limited to: forward and reverse movement of the patient by virtue of movement of the patient support structure 110 along a floor; raising and lowering movement of the patient by virtue of movement of the patient support structure 110 upward and downward relative to the floor; angular movement by virtue of changing the angle of at least a portion of the patient support structure 110 relative to a floor; rotation of the patient along a longitudinal axis of the patient support structure 110 (while the patient support apparatus 100 remains stationary relative to the floor); or various combinations of those types of movement.

Without limitation, the actuatable devices 120 that result in the change of the position of one or more patient support surfaces 141, 161 of the patient support structure 110 may include a coordinated motion device, a patient raising device, a patient turning device, a patient centering device, a patient ingress/egress device, a lift device, a fowler adjustment device, a gatch adjustment device, a side rail engagement device, and a transport device.

It is also contemplated that the actuatable device 120 may be of the type that does not result in a change of position, orientation, and/or elevation of the patient support surfaces 141, 161. These “non-position actuatable devices” may include, but are not limited to, a patient comfort device, such as an entertainment device, a lighting device, a temperature device, a humidity device, and an aromatherapy device, as well as patient therapy devices, such as vibration therapy devices, percussion therapy devices, compression therapy devices, patient warming devices, and electrical stimulation devices. The rate of operation of these non-position actuatable devices can also be controlled by changing the frequency, tempo, rate of temperature change, rate of humidity change, intensity of therapy, etc. of the devices.

The patient support apparatus 100, as shown in FIG. 1, also includes a controller 195. In FIG. 1, the controller 195 is illustrated as being disposed within the footboard 182. However, in other embodiments, the controller 195 may be disposed on or within the headboard 181, the side rails 171, 172, 173, 174, the caregiver interfaces 183, or any other suitable component of the patient support apparatus 100.

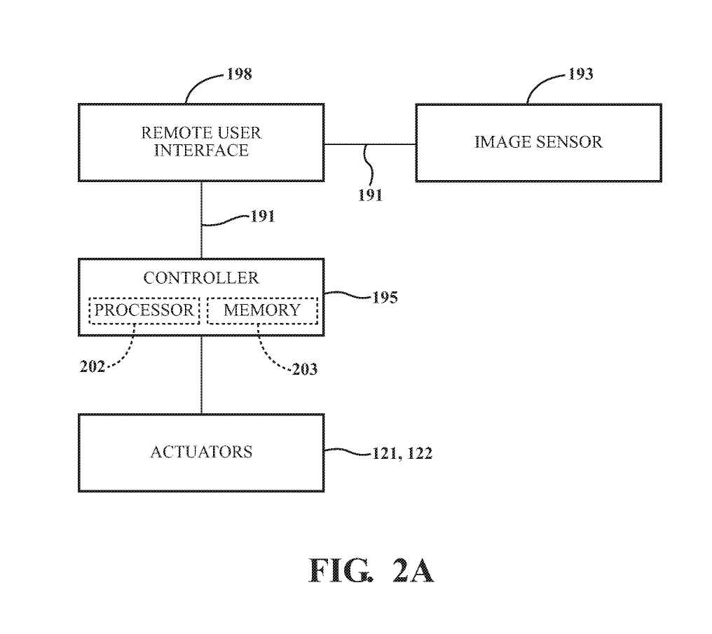

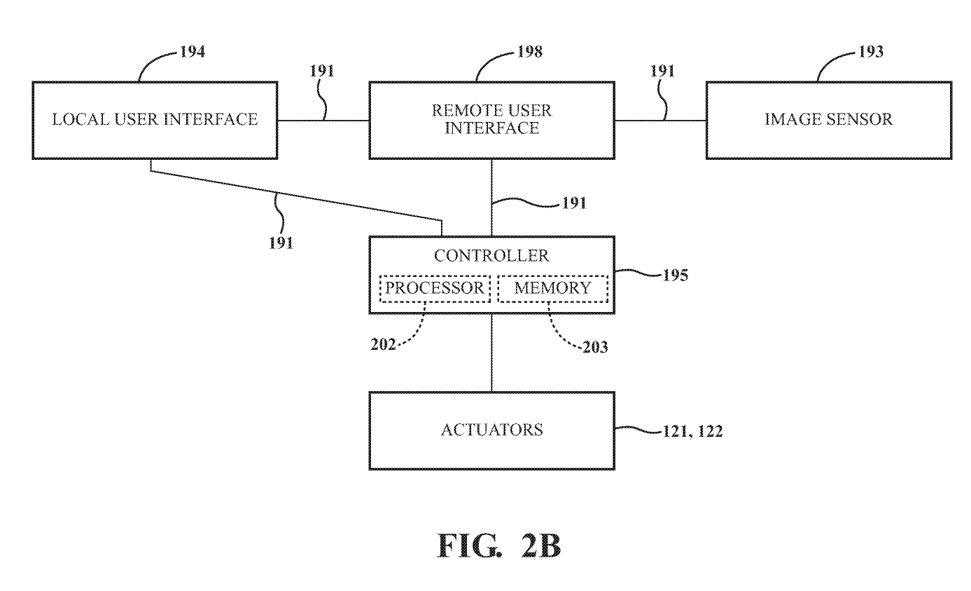

Additionally, as shown in FIGS. 2A and 2B, the controller 195 includes a processor 202 and a memory 203. The processor 202 may be any processor suitable for processing data. For example, the processor 202 may be a processor typically found in a desktop computer or a processor typically found in a mobile processing device such as a cellular phone, a tablet, or a laptop. Similarly, the memory 203 may be any memory suitable for storage of data and computer-readable instructions. For example, the memory 203 may be a local memory, an external memory, or a cloud-based memory embodied as random access memory (RAM), non-volatile RAM (NVRAM), flash memory, or any other suitable form of memory.

Referring back to FIG. 1, the system 10 may also include an image sensor 193 for capturing image data of an area including the patient support apparatus 100, referred to herein as the “patient support apparatus image data”. In the embodiment shown in FIG. 1, the image sensor 193 may be included as part of a surveillance camera. However, in other embodiments, the image sensor may be included as part of any device suitable for capturing the patient support apparatus image data, such as a digital camera, a thermographic camera, a webcam, a video camera, a livestream broadcast camera, an infrared image sensor, a visual light image sensor, or combinations thereof. Accordingly, the patient support apparatus image data may vary according to the image sensor 193. For example, if the image sensor 193 is a digital camera, the patient support apparatus image data may be a photo. In another example, if the image sensor 193 is a video camera, the patient support apparatus image data may be a video. In yet another example, if the image sensor 193 is a thermographic camera, the patient support apparatus image data may be thermal image data.

Furthermore, as shown in FIG. 1, the image sensor 193 may be coupled to a wall of the hospital room. In other embodiments, the image sensor 193 may be located in any location on a medical device or in any location suitable for capturing the patient support apparatus image data. For example, in other embodiments, the image sensor 193 may be mounted to a ceiling of the hospital room, a floor of the hospital room, or a support structure of the hospital room. In other embodiments, the image sensor 193 may be coupled to the patient support apparatus 100. For example, the image sensor 193 may be disposed on or within the headboard 181, the footboard 182, any of the side rails 171, 172, 173, 174, the caregiver interfaces 183, or any other suitable component of the patient support apparatus 100.

The system 10 may also include a remote user interface 198 for use by a remote user 196. In the embodiment shown in FIG. 1, the remote user interface 198 is a tablet device. However, the remote user interface 198 may be any suitable remote computing device. For example, the remote user interface 198 may be any one of a desktop computer or a nurse call station. In other embodiments, the remote user interface may be any suitable mobile computing device such as a cellular phone, a laptop, or a wearable remote device.

Furthermore, the remote user 196 may be any suitable persons for performing remote diagnosis of the patient support apparatus 100. For example, the remote user 196 may be a technician, a mechanic, an engineer, a calibrator, a caregiver, or any other suitable persons.

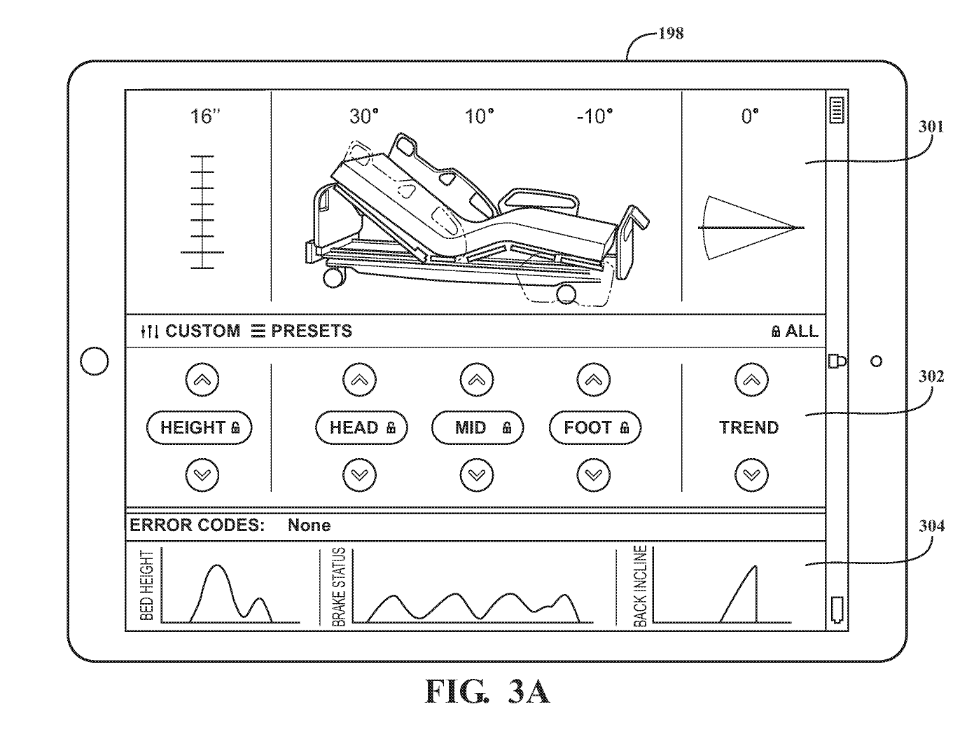

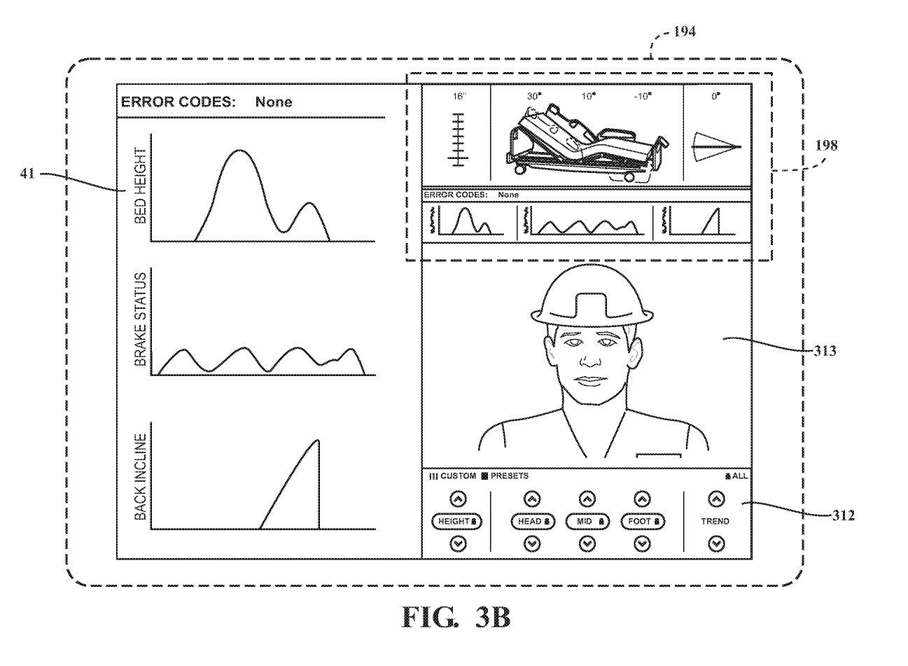

The remote user interface 198 may include a display for displaying a remote diagnostic interface 304 (shown in FIG. 3A) of a remote diagnostic application to the remote user 196. In some embodiments, the remote diagnostic interface 304 may be a graphical user interface and/or a text-based user interface.

In some embodiments, the remote user interface 198 may include a camera for capturing image data, referred to herein as the “remote image data”. For example, the remote image data may be of the remote user 196 such as a face of the remote user 196. Furthermore, the camera for capturing the remote image data may be any suitable camera, such as a digital camera, a thermographic camera, a webcam, a video camera, a livestream broadcast camera, or any other device suitable for capturing the remote image data. Additionally, the camera for capturing the remote image data may be disposed on or within the remote user interface 198. Such features may allow the remote user interface 198 to capture and transmit a video of the remote user 196.

In some embodiments, the remote user interface 198 may include a microphone for capturing audio data, referred to herein as the “remote audio data”. For example, the remote audio data may be a voice of the remote user 196. Furthermore, the microphone for capturing the remote audio data may be any suitable microphone, such as a condenser microphone, a dynamic microphone, a piezoelectric microphone, an electret microphone, a wireless microphone, or a wearable microphone. Additionally, the microphone for capturing the remote audio data may be disposed on or within the remote user interface 198. Such features may allow the remote user interface 198 to capture and transmit the voice of the remote user 196.

It should be noted that, while the embodiment shown in FIG. 1 includes one remote user interface 198, the system 10 may include any suitable number of remote user interfaces 198. For example, in some embodiments, the system 10 may also include a second remote user interface for use by a second remote user and a third remote user interface for use by a third remote user.

Furthermore, the system 10 may be designed to be used by any suitable number or types of remote users 196. For example, the system 10 may be used by a first remote user, who may be the remote user initially assigned to perform remote diagnosis on the patient support apparatus 100. In some embodiments, the system 10 may also designate a second remote user as a backup remote user to the first remote user. In other embodiments, the second remote user may be chosen from a group of available remote users. The system 10 may also appoint a master remote user, who may be assigned to monitor multiple patients. For example, the third remote user may be a nurse who is assigned to the nurse call station or who is assigned to monitor multiple patients at a local or remote command center. Of course, the system 10 may be designed for use by more than two remote users, and may include any suitable number of remote user interfaces 198.

The system 10 may also include a local user interface 194 for use by a local user 199. In some embodiments, such as the embodiment of FIG. 1, the local user interface 194 may be disposed on the patient support apparatus 100. For example, the local user interface 194 may be a user interface of the patient support apparatus 100 such as a touchscreen incorporated within an LED or LCD panel of the patient support apparatus 100. Additionally or alternatively, the local user interface 194 may be embodied as one or more buttons and/or switches of the patient support apparatus 100.

In other embodiments, the local user interface 194 may be separated from the patient support apparatus 100. For example, the local user interface 194 may be mounted to a movable cart or station positioned within a hospital room in which the patient support apparatus 100 is also positioned, a support structure of the hospital room, or a wall of the hospital room. In another example, the local user interface 194 may be a mobile computing device. For instance, the local user interface 194 may be any one of a cellular phone, a laptop, a wearable remote device, a tablet, or any other suitable mobile input device.

The local user interface 194 may include a display for displaying a local diagnostic interface 314 (shown in FIG. 3B) of a local diagnostic application to the local user 199. In some embodiments, the local diagnostic interface 314 may be a graphical user interface or a text-based user interface.

In some embodiments, the local user interface 194 may include a camera for capturing image data, referred to herein as the “local image data”. For example, the local image data may be of the local user 199 such as a face of the local user 199. Furthermore, the camera for capturing the local image data may be any suitable camera, such as a digital camera, a thermographic camera, a webcam, a video camera, a livestream broadcast camera, or any other device suitable for capturing the remote image data. Additionally, the camera for capturing the local image data may be disposed on or within the local user interface 194. Such features may allow the local user interface 194 to capture and transmit a video of the local user 199.

In some embodiments, the local user interface 194 may include a microphone for capturing audio data, referred to herein as the “local audio data”. For example, the audio data may be a voice of the local user 199. Furthermore, the microphone for capturing the local audio data may be any suitable microphone, such as a condenser microphone, a dynamic microphone, a piezoelectric microphone, an electret microphone, a wireless microphone, or a wearable microphone. Additionally, the microphone for capturing the local audio data may be disposed on or within the local user interface 194. Such features may allow the local user interface 194 to capture and transmit a voice of the local user 199.

Furthermore, the local user 199 may be any suitable person for performing local diagnosis of on the patient support apparatus 100. For example, the local user 199 may be a technician, a mechanic, an engineer, a calibrator, a caregiver, or any other suitable person.

As shown in FIG. 1, the controller 195, the remote user interface 198, and the image sensor 193 may be coupled to a communication network 191 to communicate wirelessly with one another. The communication network 191 may be any suitable communication network. For example, the communication network 191 may include any one of Bluetooth, WiFi, Infrared, ZigBee, radio waves, cellular signals, any other suitable communication network, or combinations thereof. In some embodiments, the communication network 191 may include a networking device such as a gateway device, a router, or a repeater. In other embodiments, the controller 195, the remote user interface 198, and the image sensor 193 may communicate with each other using peer-to-peer communication.

FIG. 2A and 2B provide schematic diagrams, which illustrate two embodiments of the communication network 191. In the embodiment shown in FIG. 2A, the controller 195, the image sensor 193, and the remote user interface 198 may be coupled to one another via the communication network 191. In the embodiment shown in FIG. 2B, the controller 195, the image sensor 193, the remote user interface 198, and the local user interface 194 may be coupled to one another via the communication network 191.

In the embodiment of FIG. 2A, the remote user interface 198 may be coupled to the controller 195 via the communication network 191 and may be configured to execute the remote diagnostic application. The remote diagnostic application may provide a state of the patient support apparatus 100 for display to the remote user 196 via the remote diagnostic interface 304. In such embodiments, the state of the patient support apparatus 100 may be a real-time state of the patient support apparatus 100 and/or a real-time state of one or more components of the patient support apparatus 100. For example, the real-time state of the patient support apparatus 100 may include a real-time state of a hardware component of the patient support apparatus 100, such as a real-time state of the actuators 121, 122 or the one or more actuatable devices 120 of the patient support apparatus 100. More specifically, the real-time state of the actuators 121, 122 may include a current position of the actuators 121, 122, a current speed and direction in which the actuators are moving, or an indication that the actuators are extending or retracting a portion of the patient support apparatus 100. The real-time state of the patient support apparatus 100 may also include a real-time state of a software component of the patient support apparatus 100, such as a real-time state of a software component of the controller 195 or a software component of the local user interface 194. For example, the real-time state of a software component may include an indication that a software module involved in the activation of one of the actuators 121, 122 is unresponsive, or that a software module involved in the communication with the image sensor 193 is unresponsive. In further embodiments, the real-time state of the patient support apparatus 100 may provide error codes of the patient support apparatus 100, a height of the support frame 130 of the patient support apparatus 100, side rail states (e.g., raised/lowered), brake state (e.g., caster brakes set/not set), articulation states (e.g., back section angle), etc., and/or a state of the processor 202 or the memory 203 of the controller 195 of the patient support apparatus 100.

The real-time state of the patient support apparatus 100 and components thereof may be provided by one or more sensors of the patient support apparatus 100 and/or by the controller 195. For example, a position sensor (not shown) may detect a current position of an actuator 121, 122 and may provide a position signal to the controller 195. The controller 195 may then determine the position (or state) of the actuator 121, 122 based on the position signal. Additionally or alternatively, the controller 195 may determine the position or state of another portion of the patient support apparatus 100, such as a position of patient support surfaces 141, 161, based on the position signal received from the sensor. Similarly, the controller 195 may execute a program or may use hardware components to determine the state of one or more software modules. For example, the controller 195 may execute a monitoring program that periodically polls the status of one or more software modules to determine if the software modules are responding properly. Additionally or alternatively, the controller 195 may employ a hardware or software-based watchdog timer to periodically poll the software modules to determine if the software modules are responding properly. The controller 195 may transmit data representative of the state of the patient support apparatus 100 to the local user interface 194 and/or to the remote user interface 198 to enable the respective interface to display the state.

Additionally, in the embodiment of FIG. 2A, the controller 195 may receive an input signal from the remote user interface 198, referred to herein as the “remote input signal”, which may correspond to a selected remote control function received by the remote user interface 198. The selected remote control function may be a remote control function of the patient support apparatus 100, which may be selected by the remote user 196. As shown in FIG. 2A, the controller 195 may be coupled to the actuators 121, 122 of the patient support apparatus 100 and may transmit an output signal to the actuators 121, 122 to cause movement of the one or more actuatable devices 120 based on the remote input signal.

As previously stated, the remote input signal may correspond to the selected remote control function from the remote user interface 198. The selected remote control function may be categorized as a non-patient remote control function or a patient remote control function. A remote control function is categorized as a patient remote control function if the remote control function causes movement of the patient support apparatus 100, causes movement of one or more actuatable devices 120 that are configured to move a patient or contact a patient, or could result in a change of state of the patient support apparatus 100 that could have adverse consequences for the patient. For example, a patient remote control function may cause the actuator 121, 122 to lift the side rails 171, 172, 173, 174, apply a brake of the patient support apparatus 100, lift the patient support deck 140, or incline the back section (i.e., the head end) of the patient support deck 140. In contrast, a non-patient remote control function does not cause movement of the patient support apparatus 100. For example, a non-patient remote control function may cause the controller 195 to activate a speaker 185 of the patient support apparatus 100 to play music, activate one or more lights of the patient support apparatus 100, or activate other components of the patient support apparatus 100 which are unrelated to movement.

Furthermore, in the embodiments of FIGS. 2A and 2B, the remote user interface 198 and the controller 195 may be coupled to the image sensor 193 via the communication network 191. As such, the remote user interface 198 may receive the patient support apparatus image data from the image sensor 193 and display the patient support apparatus image data to the remote user 196.

In the embodiment of FIG. 2B, the local user interface 194 may be coupled to the controller 195 via the communication network 191. In such an embodiment, the local user interface 194 may be configured to execute the local diagnostic application. In another such embodiment, the controller 195 may be configured to execute the local diagnostic application and may cause an output of the local diagnostic application to be displayed on the local user interface 194. The local diagnostic application provides the previously-defined state of the patient support apparatus 100 for display to the local user 199 via the local diagnostic interface 314. As stated above, the state of the patient support apparatus 100 may include the previously-defined real-time state of the patient support apparatus 100.

It is to be noted that, in some embodiments, the controller 195 may be coupled to the local user interface 194 without using the communication network 191. For example, in an embodiment where the local user interface 194 is the user interface of the patient support apparatus 100, such as the embodiment of FIG. 1, the local user interface 194 may be in direct communication with controller 195.

In the embodiment of FIG. 2B, the controller 195 may receive an input signal from the local user interface 194, referred to herein as the “local input signal”, which corresponds to a selected local control function received by the local user interface 194. The local remote control function may be a local control function of the patient support apparatus 100, which may be selected by the local user 199. As shown in FIG. 2B, the controller 195 may be coupled to the actuators 121, 122 of the patient support apparatus 100 and may transmit an output signal to the actuators 121, 122 to cause movement of the one or more actuatable devices 120 based on the local input signal.

Also previously stated, the local input signal corresponds to the selected local control function from the local user interface 198. Similar to the selected remote control function, the selected local control function may also be categorized as a non-patient local control function or a patient local control function. More explicitly stated, a local control function may also be categorized as a patient local control function if the local control function causes movement of the patient support apparatus 100, causes movement of one or more actuatable devices 120 that are configured to move a patient or contact a patient, or could result in a change of state of the patient support apparatus 100 that could have adverse consequences for the patient.

Furthermore, in the embodiment of FIG. 2B, the remote user interface 198 may be coupled to the local user interface 194 via the communication network 191. As such, the remote user interface 198 may transmit the remote image data and/or the remote audio data to the local user interface 194 via the communication network 191. Similarly, the local user interface 194 may transmit the local image data and/or the local audio data to the remote user interface 198. Additionally, because the local user interface 194 is coupled to the remote user interface 198 via the communication network 191, the local user interface 194 may be configured to display a portion of the remote diagnostic interface 304 of the remote diagnostic application.

FIG. 3A provides an example embodiment of the remote user interface 198 and the remote diagnostic interface 304. As shown, the remote user interface 198 is a tablet device, which displays the remote diagnostic interface 304 to the remote user 196 (not shown in FIG. 3A). In the embodiment of FIG. 3A, the remote diagnostic interface 304 is a graphical user interface that displays the state of the patient support apparatus 100. Alternatively, the remote diagnostic interface 304 may be a text-based interface or a combination of a text-based and a graphical user interface. As shown, the remote diagnostic interface 304 displays error codes of the patient support apparatus 100, a height of the support frame 130 of the patient support apparatus 100, a status of a brake of the patient support apparatus 100, as well as an incline of the back section of the patient support apparatus 100. Also shown in FIG. 3A, the remote user interface 198 also displays an interface 302 for receiving the selected remote control function from the remote user 196. Additionally, in FIG. 3A, the remote user interface 198 displays the patient support image data 301 from the image sensor 193. In other embodiments, the remote user interface 198 may display a graphical representation of the patient support apparatus 100, which may update to reflect changes in the patient support apparatus 100. In addition, the remote user interface 198 may display a log of diagnostic information reported by the controller 195. The log of diagnostic information may include data representative of other states of the patient support apparatus 100 and/or components thereof, including what actions have been performed by the local user 199 and/or the remote user 196 and the response of the patient support apparatus 100 to the actions performed by the users. For example, the log may display the response of the patient support apparatus 100 to the local and/or remote control functions executed by the local and/or remote users, including whether the functions were executed properly and a final state of the patient support apparatus 100 after the functions were executed.

The remote user interface 198 may thus display a variety of data in an efficient and convenient “split screen” manner. For example, the remote user interface 198 may display diagnostic information in one portion of the interface 198, the interface 302 for selecting a remote control function in another portion of the interface 198, a graphical representation of the patient support apparatus 100 in another portion of the interface 198, and/or the log of diagnostic information in another portion of the interface 198.

It should be noted that the remote user interface 198 shown in FIG. 3A is only one example of a remote user interface 198 that may be used. As such, the remote user interface 198 may display data and information regarding the state of the patient support apparatus 100, which is not shown in FIG. 3A. For instance, the remote diagnostic interface 304 may display a status of the side rails 171, 172, 173, 174 of the patient support apparatus 100. In other embodiments, the remote user interface 198 may optionally display the interface 302 for receiving the selected remote control function from the remote user 196. In another embodiment, the remote user interface 198 may optionally display the patient support image data 301 from the image sensor 193.

In this way, because the remote user interface 198 is coupled to the controller 195 of the patient support apparatus 100 via the communication network 191, the remote user 199 may perform remote diagnosis of the patient support apparatus 100. In some embodiments, the remote user 196 may use the remote user interface 198 to connect to the controller 195 via Bluetooth. In such embodiments, the remote user 196 may perform remote diagnosis of the patient support apparatus 100 from a different room of the hospital or within a vicinity of the patient support apparatus 100. However, in other embodiments, the remote user 196 may connect the remote user interface 198 to the controller 195 via WiFi. In such embodiments, the remote user 196 may perform remote diagnosis on the patient support apparatus 100 from any area with a WiFi connection. Advantageously, the remote user 196 may diagnose the patient support apparatus 100 from a remote location.

FIG. 3B provides an example embodiment of the local user interface 194 and the local diagnostic interface 314. The local user interface 194 may be displayed on a tablet device or may be disposed on the patient support apparatus 100. The local user interface 194 displays the local diagnostic interface 314 to the local user 199 (shown in FIG. 1). The local diagnostic interface 314, in turn, is a graphical user interface that displays the state of the patient support apparatus 100. Alternatively, the local diagnostic interface 314 may be a text-based interface or a combination of a text-based and a graphical user interface. As shown, the local diagnostic interface 314 displays error codes of the patient support apparatus 100, a height of the support frame 130 of the patient support apparatus 100, a status of a brake of the patient support apparatus 100, as well as an incline of the back section of the patient support apparatus 100. Also shown in FIG. 3B, the local user interface 194 displays an interface 312 for receiving the selected local control function from the local user 199. In addition, the local user interface 194 may display a log of diagnostic information reported by the controller 195. The log of diagnostic information may include data representative of other states of the patient support apparatus 100 and/or components thereof, including what actions have been performed by the local user 199 and/or the remote user 196 and the response of the patient support apparatus 100 to the actions performed by the users. For example, the log may display the response of the patient support apparatus 100 to the local and/or remote control functions executed by the local and/or remote users, including whether the functions were executed properly and a final state of the patient support apparatus 100 after the functions were executed.

As shown in FIG. 3B, the local user interface 194 may display the remote image data 313 and may provide the remote audio data to the local user 199. In such embodiments, the remote user 196 may provide instruction to the local user 199 concerning techniques for performing remote diagnosis and/or remote control of the patient support apparatus 100. For example, the remote user 196 may ask the local user 199 to check if a screw is loose on the patient support apparatus 100, to restart the patient support apparatus 100, or to control the patient support apparatus 100 in the event that the remote user 196 is unable to remotely control the patient support apparatus 100.

Additionally, as shown in FIG. 3B, the local user interface 194 may provide or replicate a portion of the remote user interface 198. It should be noted that, in embodiments where the local user interface 194 and the remote user interface 198 are coupled via a communication network, the remote user interface 198 may also display a portion of the local user interface 194.

The local user interface 194 may thus display a variety of data in an efficient and convenient “split screen” manner in a similar manner as described above with reference to the remote user interface 198. For example, the local user interface 194 may display diagnostic information in one portion of the interface 194, a replicated portion of the remote user interface 198 in another portion of the interface 194, the interface 312 for receiving the selected local control function in another portion of the interface 194, and/or the log of diagnostic information in another portion of the interface 194.

It should be noted that the local user interface 194 shown in FIG. 3B is only one example of a local user interface 194 that may be used. As such, the local user interface 194 may display data and information regarding the state of the patient support apparatus 100, which is not shown in FIG. 3B. For instance, the local diagnostic interface 314 may display a status of the side rails 171, 172, 173, 174 of the patient support apparatus 100. The local diagnostic interface 314 may also display data and information regarding the state of the patient support apparatus 100, which is not being displayed by the remote user interface 198. Furthermore, the local user interface 194 may exclude features and data shown in FIG. 3B. For example, in some embodiments, the local user interface 194 may optionally display the interface 312 for receiving the selected local control function from the local user 199.

Additionally, in embodiments where the remote user interface 198 may also be configured to display a graphical user interface, the graphical user interface of the local user interface 194 may be identical to the graphical user interface of the remote user interface 198. In other embodiments, the local remote user interface 194 may replicate at least a portion of the graphical user interface of the remote user interface 198.

In this way, because the local user interface 194 is coupled to the remote user interface 198 via the communication network 191, the remote user 196 may communicate with the local user 199. As such, the remote user 196 may remotely direct a local user 199 to perform diagnosis of the patient support apparatus 100 and/or to control the operation of the patient support apparatus 100. In some embodiments, the local user may be an individual not trained to perform diagnosis of the patient support apparatus 100, such as a caregiver or a nurse. In such embodiments, the local user 199 may receive instruction from the remote user 196 via the local user interface 194. In other embodiments, the remote user 196 may remotely direct a local user 199 to perform a task, which may not be accomplished by a remote user 196. For example, the remote user 196 may request that the local user 199 remove, adjust, or replace a hardware component of the patient support apparatus 100, such as a side rail or a foot board of the patient support apparatus 100.

It should be understood that, while embodiments discussed herein describe techniques for performing remote diagnosis of patient support apparatuses 100, the techniques for performing remote diagnosis may be applied to other medical devices. For instance, these medical devices may include equipment such as lights, televisions, temperature management systems, respirators, IV lines, heart rate monitors, surgical tools, or any other devices that may be used in medical procedures or in the provision of medical services to patients. Therefore, the techniques for performing remote diagnosis may be used to perform remote diagnosis of any of the above-described medical devices, or any other medical device that may be used in medical procedures or in the provision of medical services to patients.

It will be further appreciated that the terms “include,” “includes,” and “including” have the same meaning as the terms “comprise,” “comprises,” and “comprising.” Moreover, it will be appreciated that terms such as “first,” “second,” “third,” and the like are used herein to differentiate certain structural features and components for the non-limiting, illustrative purposes of clarity and consistency.

Several configurations have been discussed in the foregoing description. However, the configurations discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

1. A system for performing remote diagnosis of a medical device, the system comprising:
a medical device comprising:
an actuator configured to move a portion of the medical device; and
a controller coupled to a communication network; and
a remote user interface coupled to the communication network and being configured to:
execute a remote diagnostic application, wherein the remote diagnostic application is configured to provide a state of the medical device;
receive a selected remote control function from a user of the remote user interface; and
transmit an input signal corresponding to the selected remote control function to the controller of the medical device, wherein the controller is configured to transmit an output signal to the actuator to execute the selected remote control function based on the input signal.

2. The system as set forth in claim 1, wherein the remote user interface is a mobile device.

3. The system as set forth in claim 1, wherein the state of the medical device includes a response of the medical device to the executed remote control function.

4. The system as set forth in claim 3, wherein the state of the medical device is a real-time state of the medical device.

5. The system as set forth in claim 1, further comprising an image sensor coupled to the communication network and being configured to capture image data, wherein the image data is of an area including the medical device.

7. The system as set forth in claim 5, wherein the remote user interface comprises a display, and wherein the image data is displayed to the user of the remote user interface.

6. The system as set forth in claim 5, wherein the image sensor comprises an infrared image sensor, a visual light image sensor, or a combination thereof.

8. The system as set forth in claim 5, wherein the image sensor is coupled to the medical device.

9. The system as set forth in claim 5, wherein the image sensor is coupled to a support structure near the medical device.

10. The system as set forth in claim 1, wherein the remote user interface comprises a display, and wherein a remote diagnostic interface of the remote diagnostic application is displayed to the user of the remote user interface.

11. The system as set forth in claim 10, wherein the remote diagnostic interface is displayed in a first portion of the display and an interface for receiving the selected remote control function is displayed in a second portion of the display.

12. The system as set forth in claim 1, wherein the medical device is a patient support apparatus comprising a support structure configured to support a patient.

13. The system as set forth in claim 1, wherein the remote user interface comprises a microphone configured to capture voice data of the user of the remote user interface and a camera configured to capture image data of the user of the remote user interface.

14. A method for performing remote diagnosis of a medical device including an actuator configured to move a portion of the medical device, and a controller coupled to a communication network, the method comprising steps of:
providing a remote user interface coupled to the communication network;
executing, with the remote user interface, a remote diagnostic application;
providing a local user interface coupled to the communication network;
receiving, with the local user interface, a selected local control function from a user of the local user interface;
transmitting, with the local user interface, an input signal corresponding to the selected local control function to the controller of the medical device;
transmitting, by the controller, an output signal to the actuator to execute the selected local control function based on the input signal; and
displaying a response of the medical device to the executed local control function using the remote user interface.

15. The method as set forth in claim 14, further comprising steps of:
providing an image sensor couple to the communication network;
capturing, with the image sensor, image data, wherein the image data is of an area including the medical device.
displaying, with the remote user interface, the image data to the user of the remote user interface.

16. The method as set forth in claim 14, further comprising a step of displaying a remote diagnostic interface of the remote diagnostic application to a user of the remote user interface.

17. The method as set forth in claim 14, wherein the local user interface is coupled to the communication network and the method further comprises a step of displaying, with the local user interface, at least a portion of a remote diagnostic interface of the remote diagnostic application.

18. The method as set forth in claim 14, further comprising steps of:
executing, with the controller, a local diagnostic application; and
displaying, with the local user interface, a local diagnostic interface of the local diagnostic application to the user of the local user interface.

19. The method as set forth in claim 14, wherein the local user interface is coupled to the communication network and the method further comprises a step of:
executing, with the local user interface, a local diagnostic application; and
displaying, with the local user interface, a local diagnostic interface of the local diagnostic application to the user of the local user interface.
